(19) United States
(12) Patent Application Publication
(10) Pub. No.: US 2025/0041051 A1
(43) Pub. Date: Feb. 6, 2025

(54) MIDDLE TURBINATE MEDIALIZER AND METHOD OF USE

(71) Applicant: Todd Christopher Morehouse, Blue Bell, PA (US)

(72) Inventor: Todd Christopher Morehouse, Blue Bell, PA (US)

(21) Appl. No.: 18/787,275

(22) Filed: Jul. 29, 2024

Related U.S. Application Data
(60) Provisional application No. 63/530,357, filed on Aug. 2, 2023.

(51) Int. Cl. A61F 2/18; A61B 17/00; A61B 17/24; A61B 17/34

(52) U.S. Cl. CPC A61F 2/186; A61B 17/24; A61B 17/3468; A61B 2017/00991; A61F 2210/0004; A61F 2220/0008; A61F 2250/0065

(57) ABSTRACT

A device for medializing nasal tissue is configured to deploy an implant across at least one turbinate and a septum. In an embodiment, an implant dart comprises anchors on both ends to hold turbinates against a septum. The implant dart can be bioabsorbable. The implant dart can be delivered from a trocar positioned with a deployment tool before the trocar pierces the nasal tissue, and the implant dart is deployed from within a trocar hollow. A telescoping trocar allows positioning inside a nasal cavity through the patient's nostrils prior to deployment.

### RELATED APPLICATIONS

The present application claims benefit to U.S. Provisional Patent Application No. 63/530,357, entitled “MIDDLE TURBINATE MEDIALIZER AND METHOD OF USE,” filed on Aug. 2, 2023, which is hereby incorporated by reference in its entirety.

### BACKGROUND OF THE INVENTION

### Field of the Invention

The present general inventive concept is directed to a method and apparatus for endoscopic sinus surgery. The invention provides a device and method for medializing the middle turbinate after endoscopic surgery, preventing middle turbinate lateralization, and improving surgical outcomes.

### Description of the Related Art

The prior art discloses methods and devices for securing a middle turbinate to a nasal septum including U.S. Pat. No. 9,999,433 to Jenkins et al. The patent discloses a device for piercing a nasal septum and a middle turbinate with a barbed suture and holds the turbinate against the nasal septum. The disclosed device provides a suture with numerous barbs designed to lodge in a tissue and an anchor present at the end of the suture. The device can be used to secure one or more middle turbinates (medialize the turbinate) to the septum by inserting a needle and suture on one side of the septum and grasping the needle with a grasping tool to pull the suture and barbs until an anchor at the tail end of the suture engages the turbinate against the septum. The excess length of the barbed suture needs to be severed flush with the surface of the septum. Alternate embodiments employ a slip knot to pull the barbed suture and anchor together to pull turbinates against a septum before trimming or severing an excess amount of a free end of the suture or a needle utilized in the procedure. This approach requires surgical actions akin to stitching utilizing a needle, suture, and trimming excess length or utilizing a knot. Because each turbinate is not positioned against the septum at the start of the procedure, excess length of the barbed suture is contemplated to reach across the separation and then reduction of the length is required after medializing has been achieved.

U.S. Pat. No. 8,070,032 to Tagge discloses methods and devices for medializing middle turbinates. The patent discloses a method of using a forked device for insertion into the nose that can be activated to staple the turbinates to the septum. An additional embodiment contemplates using a rivet to medialize a turbinate to a nasal septum. Additional disclosure suggests the use of adhesive deployed through hollow rods to attach turbinates to a septum. The fastener staple suggested in Tagge is more quickly deployed, however, it does not provide the retention function of an anchor to prevent movement of the fastener. The nasal passages are especially subject to extreme forces for example during sneezing as compared to other parts of the body where surgical sutures and staples can be expected to remain in place. Sutures, stitches, and staples applied to external tissue can be bandaged or taped to avoid disruption. The inclusion of materials in the nasal cavity can lead to complications including infection and can hamper breathing.

What is needed is a device for medializing the middle turbinates to the nasal septum that can be rapidly deployed, accurately placed, avoid the need for additional stitching or severing of materials, and can anchor or secure the turbinates to the septum in a durable manner.

### SUMMARY OF THE INVENTION

It is an object of the present invention to provide an expandable implant that can be deployed from a trocar. The trocar pierces an opening through a pair of turbinates and a patient's septum before deploying an implant dart with a retaining anchor on both sides that is configured to bind the turbinates against the septum and medialize the middle turbinates.

In an aspect of the invention is disclosed a surgical device for attaching a turbinate to a septum comprising an implant comprising a telescoping stem, a proximal anchor connected to a proximal end of said telescoping stem, a distal anchor connected to a distal end of said telescoping stem, said proximal anchor is configured to deploy to an increased radial diameter, and said distal anchor is configured to deploy to an increased radial diameter. In another aspect of the invention, a method is provided for utilizing the surgical device for medializing a turbinate in a human patient comprising utilizing the surgical device to secure a turbinate and a septum in a nasal cavity.

Another aspect of the invention discloses an implantable surgical device for affixing sinus tissue comprising a deployment tine configured for insertion into a nasal cavity and positioning a socket transverse to a direction of insertion, a trocar base disposed inside said socket, a trocar body disposed inside said trocar base, said trocar body connected to a trocar extender wherein movement of said trocar extender into said socket deploys said trocar body out of said socket, said trocar body having a bladed trocar distal end; an implant retained inside said trocar body by friction fit, said implant comprising a telescoping stem, a proximal anchor, and a distal anchor; a countertraction tine configured for insertion into a second nasal cavity and positioning a countertraction arm on an opposite side of a septum from said socket, said countertraction tine being movable to adjust a distance to said deployment tine; an implant extender configured to move said implant out of said trocar body wherein said distal anchor deploys and extends to a diameter greater than said bladed trocar distal end; and wherein movement of said trocar extender out of said socket retracts said trocar body into said socket allowing said proximal anchor to deploy and extend to a diameter greater than said bladed trocar distal end. Another aspect of the invention discloses a method medializing middle of medializing turbinates in a human patient comprising utilizing the device to deploy the implant across a first turbinate, the septum, and a second turbinate.

A deployment tool is provided that holds the trocar in a socket and provides for selective deployment of the trocar and the implant dart as well as selective retraction of the trocar and the implant dart.

In a method of utilizing the structure of the invention, the deployment tool is used to compress the turbinates against the septum and deploy the trocar to pierce the patient first turbinate, septum, and second turbinate and deliver an implant through the opening created by the trocar. A distal anchor is deployed on the outer side of the second turbinate before withdrawing the trocar and expandable implant towards the socket to deploy a proximal anchor on the outer side of the first turbinate.

These together with other aspects and advantages which will be subsequently apparent, reside in the details of construction and operation as more fully hereinafter described, reference being had to the accompanying drawings forming a part hereof, wherein like numerals refer to like parts throughout.

### BRIEF DESCRIPTION OF THE DRAWINGS

Further features and advantages of the present invention, as well as the structure and operation of various embodiments of the present invention, will become apparent and more readily appreciated from the following description of the preferred embodiments, taken in conjunction with the accompanying drawings of which:

FIG. 1 is a perspective view of an implant dart in an expanded configuration.

FIG. 2 is a side view of an implant dart partially disposed within a trocar, and a socket.

FIG. 3 is a perspective view of a trocar and a socket in a compressed configuration.

FIG. 4 is a sectional view of an implant dart disposed within a trocar inside a socket.

FIG. 5 is a sectional view of an implant dart positioned within an extended trocar.

FIG. 6 is a sectional view front view of an implant dart partially deployed from a trocar body.

FIG. 7 is a front view of a deployment tool positioned around turbinates in a nasal cavity.

FIG. 8 is a front view of a trocar piercing turbinates pressed against a septum.

FIG. 9 is a front view of a patient sinus cavity with an implant dart deployed on both sides of turbinates bound to a septum.

### DESCRIPTION OF THE PREFERRED EMBODIMENTS

Reference will now be made in detail to the presently preferred embodiments of the invention, examples of which are illustrated in the accompanying drawings, wherein like reference numerals refer to like elements throughout.

The present inventive concept relates to a device for facilitating sinus surgery. In particular, the invention provides an apparatus for rapid and consistent medialization of nasal turbinates to the nasal septum in a human nose. The device consists of a deployment tool suited to position a socket 170 that is mounted transverse to the direction of insertion into the nasal cavity. The socket 170 can be permanently or removably connected to a deployment tine 210. The socket 170 is suited to retain a trocar comprising a trocar base 160 and a trocar body 150. In an embodiment, trocar distal end 155 provides additional telescoping distance to pierce both turbinates, the septum, and allow for deployment of an implant such as implant dart 100 in a sinus cavity. The device is configured with telescoping sections so that it can be inserted into the nasal cavity in limited space and then extend across the septum and turbinates to achieve medialization. Trocar body 150 is configured to extend from trocar base 160 and retract back into trocar base 160 and socket 170.

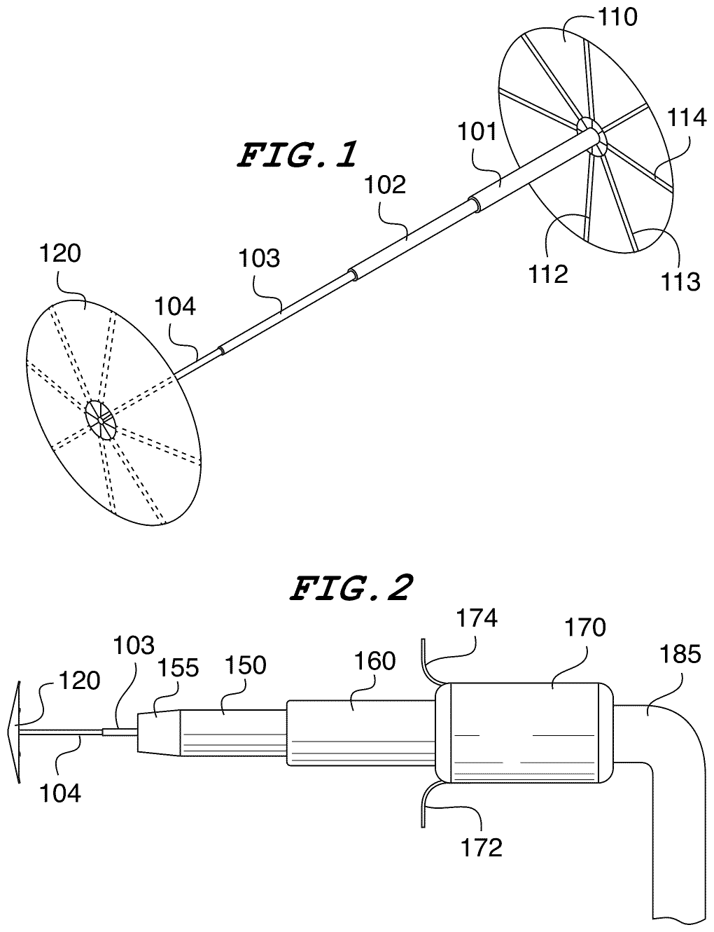

FIG. 1 shows implant dart 100. Implant dart 100 can be thin or needle shaped when retracted and can have two umbrella shaped anchors, proximal anchor 110 and distal anchor 120. Telescoping stem 101 connects the anchors and is configured to receive the several telescoping elements first extension 102, second extension 103, and third extension 104. Implant 100 can be made of bioabsorbable materials such as Vicryl or other suitable materials. Proximal anchor 110 is shown at the proximal end of telescoping stem 101 and can comprise a circular shape with thickened ribs that are resilient and biased towards an expanded configuration to help deploy the anchor when it is no longer constrained inside the trocar. For example, first rib 112, second rib 113, and third rib 114 are shown disposed on proximal anchor 110 in FIG. 1 and extend radially from telescoping stem 101. Each anchor will self-deploy or expand when not constrained. Distal anchor 120 is shown at the distal end of telescoping stem 101 and in an embodiment can be connected to third extension 104 and can be configured to deploy automatically when not compressed. A resilient implant dart 100 can be folded and loaded into the trocar shortly before use on a patient. Removal from a restriction such as trocar body 150 will allow the anchors to spring back to the configuration shown in FIG. 1 where proximal anchor 110 is shown deployed with an increased radial diameter and distal anchor 120 is shown deployed with an increased radial diameter.

FIG. 2 presents a view of the implant, here implant dart 100 partially deployed from trocar body 150. Socket 170 is shown partially surrounding trocar base 160 with trocar body 150 extended. In an embodiment trocar blade 156 and trocar blade 157 can be disposed on the distal end of trocar distal end 155. These blades can be separate or continuous. In an alternate embodiment, the distal end of the trocar distal end 155 can be pointed or sharpened or comprise exterior blades. Socket 170 is connected to deployment arm 185 which directs activation elements into the socket and can be a hollow tube made of metal or plastic. Activation elements can comprise trocar extender 180 and implant extender 190. In an embodiment trocar extender 180 is configured to be coaxial with implant extender 190 and comprises a flexible but incompressible material such as metal. In utilizing the device, trocar extender 180 can be fixedly attached to trocar body 150 so that movement of trocar extender 180 into and out of the socket 170 moves the trocar body 150 a corresponding distance. In an embodiment, implant extender 190 is not fixedly connected to the implant 100 so that it can push the implant into the patient tissue, but removal of the implant extender 190 does not retract the implant 100 out of the patient or into the trocar body 150. FIG. 2 shows that the distal anchor 120 has been pushed out of the trocar body 150 and it has expanded or deployed like an umbrella into a significant sized area compared to the width of third extension 104.

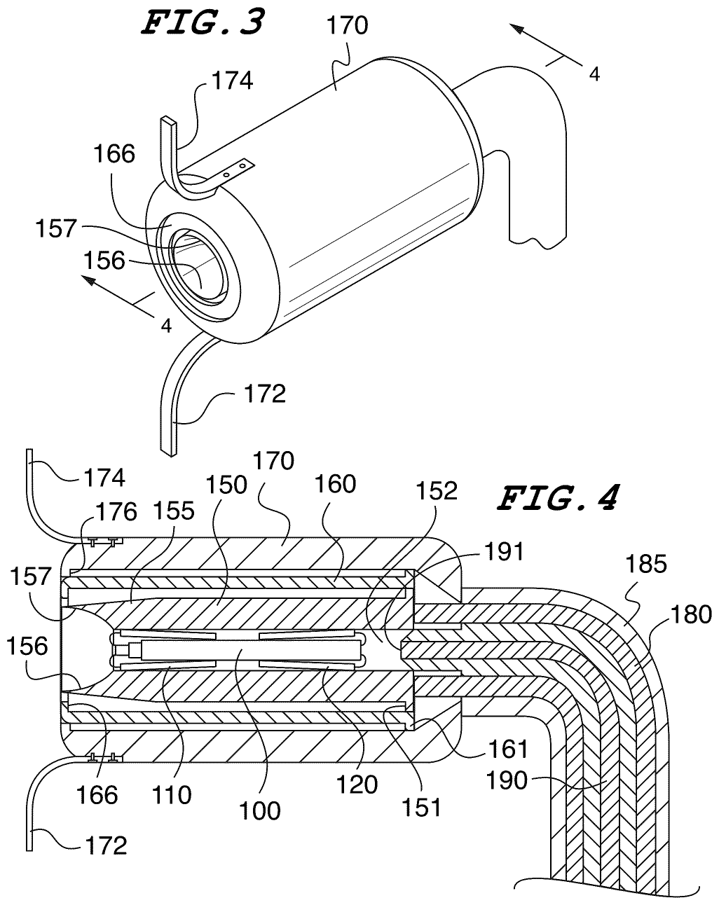

FIG. 3 shows a perspective view of a trocar in socket 170. In order to provide sufficient space for proximal anchor 110 to deploy, socket 170 can comprise socket arm 172 and second socket arm 174 to press first turbinate 10 against septum 30 in position for medialization.

FIG. 4 presents a sectional view of the implant dart 100 of the invention loaded into trocar hollow 152 centrally disposed within trocar body 150. The proximal anchor 110 is shown compressed around telescoping stem 101. The distal anchor 120 is shown compressed around telescoping stem 101. Trocar extender 180 can be pushed to extend trocar body 150 and trocar base 160 out of socket 170. Body shoulder 151 engages base collar 166 to draw trocar base 160 out of socket 170. Body shoulder 151 can be a circumferential structure that extends outwardly from trocar body 150 in a complete circumference, or can comprise discrete tabs, in order to engage base collar 166. Base collar 166 can also be a circumferential structure that projects inwardly to cooperate with body shoulder 151. After blades 156 and 157 have cleared the second turbinate 20, implant extender 190 can push implant 100 out of the recess of trocar body 150 to deploy distal anchor 120. Extender distal end 191 contacts implant 100. Socket collar 176 comprises a reduced radial diameter relative to socket 170 and maintains maximum extension of trocar base 160 out of socket 170 by contact with the increased radial diameter of base shoulder 161.

Base shoulder 161 engages socket collar 176 to limit the travel of trocar base 160 out of socket 170 and prevents the two structures from disconnecting. Base shoulder 161 can be a circumferential structure that extends outwardly from trocar base 160 in a complete circumference, or can comprise discrete tabs or a partial circumference. Socket collar 176 can also be a circumferential structure that projects inwardly to cooperate with base shoulder 161.

The trocar extender 180 can be coaxial with the implant extender 190. The trocar extender 180 is resilient and preferably incompressible to push the trocar body 150 forward and can be fixedly connect to trocar body 150. In alternate embodiments, the implant dart can be deployed with other mechanisms or impulse forces such as compressed air combined with a countertraction tine 220 that provides a conical stopping point (not shown) to arrest the implant dart and allow for full extension of distal anchor 120.

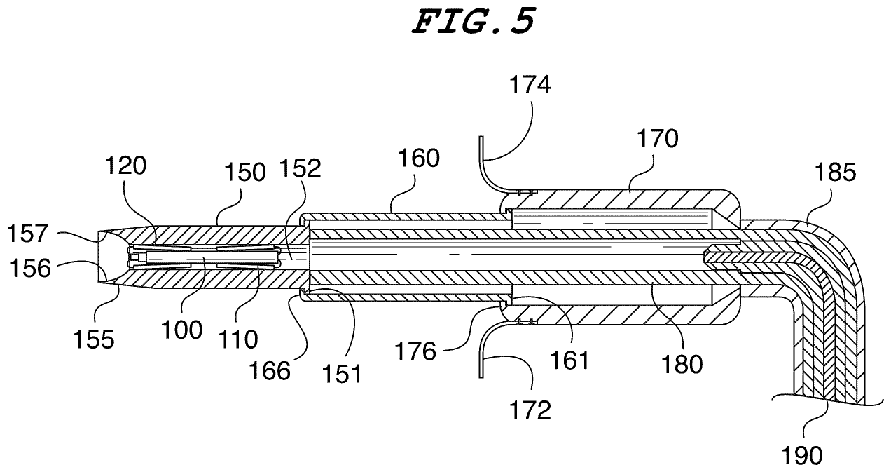

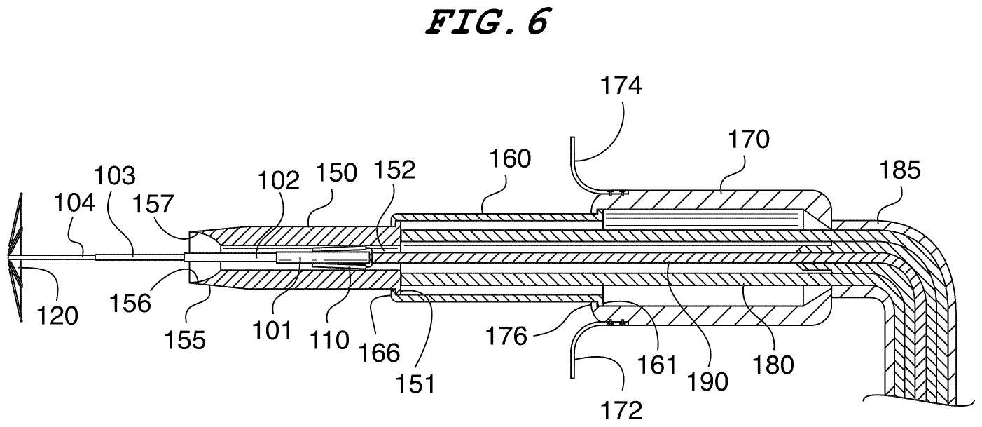

FIG. 5 shows the trocar body 150 fully extended with trocar base 160 fully extended. FIG. 6 shows the implant dart 100 pushed out of trocar body 150 by the movement of implant extender 190. With distal anchor 120 deployed on the outside of a second turbinate 20, removal of trocar body 150 by retraction of trocar extender 180 draws both trocar body 150 and trocar base 160 into socket 170. Implant dart 100 will be limited in movement by distal anchor 120. Implant dart 100 can also be kept in place selectively by manipulation of implant extender 190 during retraction if desirable. The length of trocar base 150 and trocar body 160 in relation to implant dart 100 when fully extended can all be adjusted so that proximal anchor 110 deploys on the outside of the first turbinate 10 and within the space created by socket arm 172 and socket arm 174 between the first turbinate 10 and the socket 170.

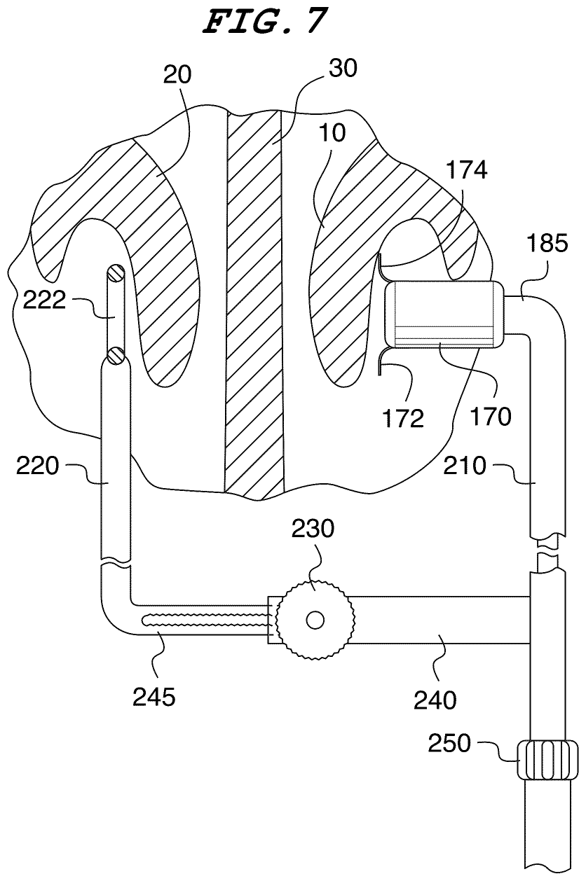

The invention provides a deployment tool for positioning and securing the implant to a patient. FIG. 7 shows countertraction tine 220 comprises countertraction arm 222 that can be a circular element, or a semicircular element, and is configured to allow the deployment of distal anchor 120 within the circumference of countertraction arm 222 when it holds second turbinate 20 against septum 30. Socket 170 is shown positioned adjacent turbinate 10. As the nasal cavity is generally symmetrical with a middle turbinate on each side of the septum 30, it is contemplated that the implant can be deployed from right to left, or from left to right, interchangeably. The deployment tool could be flipped over and deployed in reverse. As shown, the countertraction tine 220 is connected to arm 245 which is notched to be moved by turning width adjustment wheel 230 to draw arm 245 into and out of sleeve 240 which is connected to deployment tine 210. In an embodiment, arm 245 has a cross-sectional area (such as square) that corresponds to the cross-sectional area within sleeve 240 to maintain a smooth and rigid retention of the countertraction tine 220 relative to deployment tine 210. A tight tolerance can create a friction fit to maintain relative positions and a larger width adjustment wheel 230 can provide increased leverage to more easily adjust the relative positions. In an embodiment, a depth adjustment wheel 250 can be provided that adjusts the depth of the two tines relative to the sinus cavity of the patient and can be held in place by a fixed structure. Optionally, the device can be moved in and out of the sinus cavity manually. Trocar extender 180 can utilize a teeth and groove system (not shown) and a sufficient size and mechanical advantage to provide the force needed to pierce a septum and turbinates with trocar blade 156 in an embodiment of the invention. In an alternate embodiment of the invention, a spring loaded trigger can be utilized to deploy trocar extender 180 rapidly and force trocar distal end 155 through the patient tissue. Compressed air, mechanical advantage, or manual force can be employed to move trocar extender 180 through deployment arm 185 to pierce nasal structures.

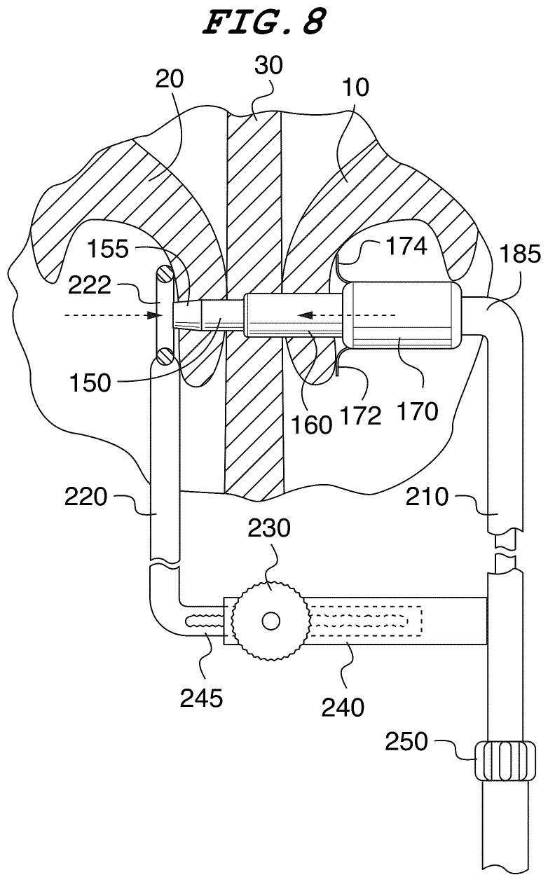

FIG. 8 shows first turbinate 10 pressed against septum 30 by socket arm 172 and second socket arm 174. Second turbinate 20 is pressed against septum 30 by countertraction arm 222. The trocar blades 156 and 157 have pierced first turbinate 10, septum 30, and second turbinate 20 to allow the center of distal anchor 120 to be positioned within countertraction arm 222. The telescoping elements of the trocar are sufficient in length to pierce the septum and both turbinates. In an alternate embodiment trocar distal end 155 can comprise one or more blades along a portion of its length, or can alternately be bladed along its entire length with blades that protrude from the angled surface, but do not interfere with the retraction of trocar distal end 155 into trocar body 150 or trocar base 160.

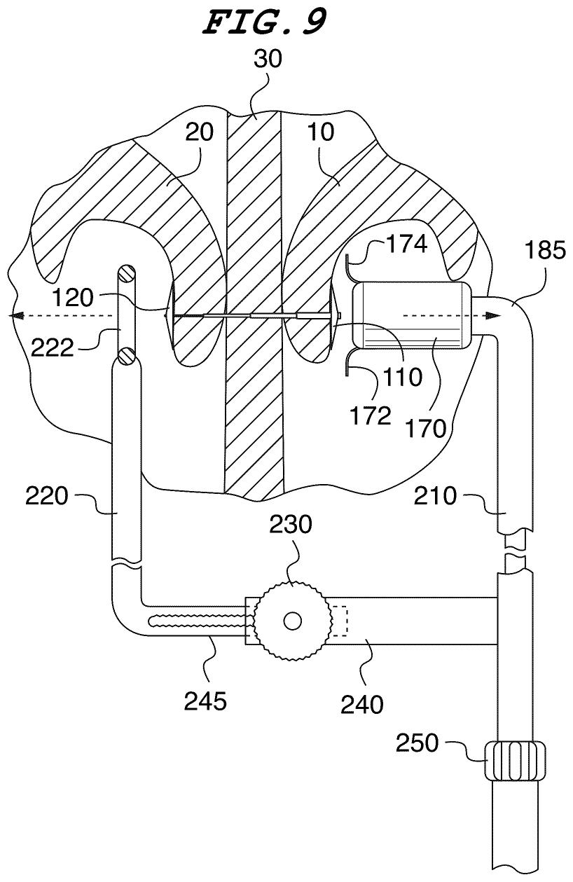

FIG. 9 shows the distal anchor 120 deployed outside the second turbinate 20. After the trocar blades have pierced second turbinate 20, implant extender 190 is pushed forward to extend implant 100 out of the trocar hollow 152. After distal anchor 120 is deployed, implant extender 190 can be optionally withdrawn. It is expected that proximal anchor 110 will maintain its folded and telescoped configuration inside the trocar hollow 152 of trocar body 150 and be drawn by friction fit towards socket 170 until distal anchor 120 contacts the exterior of second turbinate 20, and then trocar body 150 will begin to pull proximal anchor 110 away from distal anchor 120 and extend telescoping stem 101 exposing fully first extension 102, second extension 103, and third extension 104, in any order. At the point of full extension, proximal anchor 110 will be drawn out of trocar body 150 and will deploy without the restriction provided by trocar body 150. Space between the first turbinate 10 and the socket 170 is provided by socket arm 172 and optionally second socket arm 174. Other embodiments such as a cone or other mechanism similar to countertraction arm 22 can provide the space between first turbinate 10 and socket 170 needed to deploy proximal anchor 110.

After both anchors have been deployed on opposite sides of the turbinates, the turbinates have been successfully medialized against the septum. In a method of utilizing the invention, the inside surfaces of the middle turbinates and the adjacent surface of the septum can be roughed in a process call Bulgerization in order to ensure scar tissue forms to permanently medialize the turbinates against the septum.

The width adjustment wheel 230 can now be utilized to spread the tines 210 and 220 apart and withdraw the device from the nasal passage, typically out through the nostrils.

The socket can be integrated into the deployment tine or alternately removable. The deployment tine 210 and countertraction tine 220 maintain the position of the subject turbinates while the trocar pierces the patient tissue. The deployment tool can be used to compress the turbinates and measure the distance to determine the needed length of implant 100 for a particular patient, prior to surgery. Markings on arm 245 can facilitate measurement units or selection of corresponding implant sizes.

In an embodiment of the invention, both the implant dart and the trocar body 150 are pushed forward by a deployment member that can be controlled remotely and outside the sinus passage. In one particular embodiment a first deployment member is attached to the base of the trocar and a second deployment member is contained at least partially coaxially with the first deployment member, is not attached to the implant dart, but is configured to push the implant dart out of the trocar a sufficient distance so that the distal anchor 120 is deployed. Retracting the trocar body 150 will draw the implant dart towards the socket 170 until the deployed distal anchor 120 engages a medial turbinate and causes the telescoping sections of the implant dart to extend out of the trocar body. As the trocar continues to be retracted, the proximal anchor 110 is exposed and is able to deploy. Resilient ribs cause the proximal anchor 110 to expand.

Additional features of the deployment tool are provided to position and activate the implant dart, trocar, and socket to ensure successful placement. It is contemplated that countertraction tine 220 will be coplanar with deployment tine 210 and fixed in orientation with each other to ensure that the distal ends are aligned. The compression of the middle turbinates can be accomplished by adjusting the countertraction tine 220 with respect to the deployment tine 210 and can be measured prior to the medialization procedure.

The trocar can be provided with a sharp, bladed end. It can be plastic with metal blade elements, or unitary metal construction, or other materials known in the art. In order to deploy a sufficient distance across the turbinates while still being small enough to fit in the nasal passage, it is desirable to deploy a telescoping trocar structured in two or three sections to cut through turbinates and septum to a desired depth. More telescoping sections can be utilized in the spirit of the invention. Trocar extender 180 can be fixed to the base of the trocar associated with the centermost section that will deploy the furthest. In this way, a force pushed upon the center section will cause the center, smallest portion of the trocar body to move forward and draw out the subsequent sections as needed to reach full extension. For example, extending trocar body 150 will cause body shoulder 151 to engage base collar 166 to draw trocar base 160 out of socket 170. Full extension is not necessary prior to deployment of the implant 100 by implant extender 190.

The trocar base 160 should have a central access opening for implant extender 190 to push the implant dart partially out of the trocar after sufficient deployment of the trocar. When the trocar has fully pierced the near turbinate, septum, and far turbinate, the implant dart will still be contained within the trocar. Moving the implant extender 190 can deploy implant dart 100 at any degree of trocar extension. It is contemplated that implant extender 190, can be smaller than the trocar extender 180 as it will only be required to push implant dart 100 against friction of the trocar body 150 and the implant dart is expected to deploy into a void space within countertraction tine 222 with little resistance. The deployment tool can comprise width adjustments and depth adjustments as well as structure to maintain both tines of the tool as coplanar and aligned for deployment into patient tissue from a first tine to a second tine or, in a particular embodiment, deployment tine 210 to countertraction tine 220.

One of the advantages of the present invention is the ability to position the turbinates into a medialized position contacting the septum prior to piercing of the nasal tissue. The device can compress and restrain the turbinates at the same time as they are both pierced, and medialized. The bladed trocar makes an opening in the nasal tissues, and then the implant is deployed with self-deploying anchors at each end. The length of the implant dart is preselected to avoid any trimming or adjustment of size or length after deployment. Implant dart 100 can be bioabsorbable wherein no follow up surgery or removal is required.

The operations described herein can be performed in any sensible order. Any operations not required for proper operation can be optional. The many features and advantages of the invention are apparent from the detailed specification and, it is intended to cover all such features and advantages of the invention that fall within the true spirit and scope of the invention. Further, since numerous modifications and changes will readily occur to those skilled in the art, it is not desired to limit the invention to the exact construction and operation illustrated and described, and accordingly all suitable modifications and equivalents may be resorted to, falling within the scope of the invention.

What is claimed is:

1. A surgical device for attaching a turbinate to a septum comprising:
an implant comprising a telescoping stem;
a proximal anchor connected to a proximal end of said telescoping stem;
a distal anchor connected to a distal end of said telescoping stem;
said proximal anchor is configured to deploy to an increased radial diameter; and
said distal anchor is configured to deploy to an increased radial diameter.

2. The surgical device of claim 1 wherein:
said telescoping stem comprises a first extension configured to be inserted into said telescoping stem, a second extension configured to be inserted into said first extension, and a third extension configured to be inserted into said second extension.

3. The surgical device of claim 1 further comprising a telescoping trocar comprising a trocar body having a trocar distal end, said trocar body configured to extend from within a trocar base, said trocar base configured to extend from a socket.

4. The surgical device of claim 3 wherein said implant is configured for insertion inside trocar hollow within said trocar body with said proximal anchor compressed around said telescoping stem and said distal anchor compressed around said telescoping stem.

5. The surgical device of claim 4 wherein:
said trocar body comprises increased radial diameter body shoulder and trocar base comprises cooperating reduced radial diameter base collar where said body shoulder contacts said base collar to limit travel of trocar body out of trocar base; and
said trocar base comprises increased radial diameter base shoulder and socket comprises cooperating reduced radial diameter socket collar where base shoulder contacts socket collar to limit travel of trocar base out of socket.

6. The surgical device of claim 5 further comprising:
a trocar extender fixedly connected to said trocar body wherein movement of said trocar extender into said socket causes movement of said trocar body in a distal direction out of said socket, and movement of said trocar extender in a proximal direction causes movement of said trocar body into said socket.

7. The surgical device of claim 6 wherein movement of said trocar body in said distal direction engages said base collar to move said trocar base in said distal direction.

8. The surgical device of claim 7 further comprising:
an implant extender configured to contact said implant wherein movement of said implant extender into said socket causes movement of said implant in a distal direction out of said trocar body to deploy said distal anchor.

9. The surgical device of claim 8 wherein said implant extender can be moved independent of said trocar extender.

10. The surgical device of claim 9 wherein said implant extender is coaxial with said trocar extender.

11. The surgical device of claim 9 wherein said implant extender is positioned interior to said trocar extender.

12. The surgical device of claim 8 wherein said implant comprises bioabsorbable materials.

13. The surgical device of claim 8 wherein said proximal anchor is configured to compress a first turbinate against a septum and said distal anchor is configured to compress a second turbinate against the septum.

14. The surgical device of claim 13 wherein said trocar distal end comprises a trocar blade configured to penetrate a first turbinate, the septum, and a second turbinate to position said trocar distal end past the second turbinate for deployment of said distal anchor.

15. The surgical device of claim 14 wherein said proximal anchor is configured to be moved in said proximal direction within said trocar body by retraction of said trocar extender, and said distal anchor is configured to draw said third extension out of said second extension.

16. The surgical device of claim 15 further comprising at least one socket arm connected to said socket and configured to space said socket from the first turbinate to provide space for deployment of said proximal anchor.

17. The surgical device of claim 16 further comprising:
a deployment tine configured to position said socket inside a nasal cavity against the first turbinate;
a countertraction tine configured to position a countertraction arm inside a second nasal cavity against the second turbinate; and
a width adjustment configured to move said deployment tine relative to said countertraction tine to effect compression of the first turbinate and the second turbinate.

18. An implantable surgical device for affixing sinus tissue comprising:
a deployment tine configured for insertion into a nasal cavity and positioning a socket transverse to a direction of insertion, a trocar base disposed inside said socket, a trocar body disposed inside said trocar base, said trocar body connected to a trocar extender wherein movement of said trocar extender into said socket deploys said trocar body out of said socket, said trocar body having a bladed trocar distal end;
an implant retained inside said trocar body by friction fit, said implant comprising a telescoping stem, a proximal anchor, and a distal anchor;
a countertraction tine configured for insertion into a second nasal cavity and positioning a countertraction arm on an opposite side of a septum from said socket, said countertraction tine being movable to adjust a distance to said deployment tine;
an implant extender configured to move said implant out of said trocar body wherein said distal anchor deploys and extends to a diameter greater than said bladed trocar distal end; and
wherein movement of said trocar extender out of said socket retracts said trocar body into said socket allowing said proximal anchor to deploy and extend to a diameter greater than said bladed trocar distal end.

19. The device of claim 18 wherein said implant is bioabsorbable, and wherein said implant is configured to adhere a first turbinate to the septum and adhere a second turbinate to the septum.

20. A method of medializing turbinates in a human patient comprising utilizing the device of claim 18 to deploy said implant across a first turbinate, the septum, and a second turbinate.
